SEQUENCE LISTING (3 sequences)

SEQ ID NO: 1 - protein, 1738 aa, Homo sapiens
MLKRKQSSRVEAQPVTDFGPDESLSDNADILWINKPWVHSLLRICAIISVISVCMNTPMTFEHYPPLQYVTFTLDTLLMFLYTAEMIAKMHIRGIVKGDSSYVKDRWCVFDGFMVFCLWVSLVLQVFEIADIVDQMSPWGMLRIPRPLIMIRAFRIYFRFELPRTRITNILKRSGEQIWSVSIFLLFFLLLYGILGVQMFGTFTYHCVVNDTKPGNVTWNSLAIPDTHCSPELEEGYQCPPGFKCMDLEDLGLSRQELGYSGFNEIGTSIFTVYEAASQEGWVFLMYRAIDSFPRWRSYFYFITLIFFLAWLVKNVFIAVIIETFAEIRVQFQQMWGSRSSTTSTATTQMFHEDAAGGWQLVAVDVNKPQGRAPACLQKMMRSSVFHMFILSMVTVDVIVAASNYYKGENFRRQYDEFYLAEVAFTVLFDLEALLKIWCLGFTGYISSSLHKFELLLVIGTTLHVYPDLYHSQFTYFQVLRVVRLIKISPALEDFVYKIFGPGKKLGSLVVFTASLLIVMSAISLQMFCFVEELDRFTTFPRAFMSMFQILTQEGWVDVMDQTLNAVGHMWAPVVAIYFILYHLFATLILLSLFVAVILDNLELDEDLKKLKQLKQSEANADTKEKLPLRLRIFEKFPNRPQMVKISKLPSDFTVPKIRESFMKQFIDRQQQDTCCLLRSLPTTSSSSCDHSKRSAIEDNKYIDQKLRKSVFSIRARNLLEKETAVTKILRACTRQRMLSGSFEGQPAKERSILSVQHHIRQERRSLRHGSNSQRISRGKSLETLTQDHSNTVRYRNAQREDSEIKMIQEKKEQAEMKRKVQEEELRENHPYFDKPLFIVGREHRFRNFCRVVVRARFNASKTDPVTGAVKNTKYHQLYDLLGLVTYLDWVMIIVTICSCISMMFESPFRRVMHAPTLQIAEYVFVIFMSIELNLKIMADGLFFTPTAVIRDFGGVMDIFIYLVSLIFLCWMPQNVPAESGAQLLMVLRCLRPLRIFKLVPQMRKVVRELFSGFKEIFLVSILLLTLMLVFASFGVQLFAGKLAKCNDPNIIRREDCNGIFRINVSVSKNLNLKLRPGEKKPGFWVPRVWANPRNFNFDNVGNAMLALFEVLSLKGWVEVRDVIIHRVGPIHGIYIHVFVFLGCMIGLTLFVGVVIANFNENKGTALLTVDQRRWEDLKSRLKIAQPLHLPPRPDNDGFRAKMYDITQHPFFKRTIALLVLAQSVLLSVKWDVEDPVTVPLATMSVVFTFIFVLEVTMKIIAMSPAGFWQSRRNRYDLLVTSLGVVWVVLHFALLNAYTYMMGACVIVFRFFSICGKHVTLKMLLLTVVVSMYKSFFIIVGMFLLLLCYAFAGVVLFGTVKYGENINRHANFSSAGKAITVLFRIVTGEDWNKIMHDCMVQPPFCTPDEFTYWATDCGNYAGALMYFCSFYVIIAYIMLNLLVAIIVENFSLFYSTEEDQLLSYNDLRHFQIIWNMVDDKREGVIPTFRVKFLLRLLRGRLEVDLDKDKLLFKHMCYEMERLHNGGDVTFHDVLSMLSYRSVDIRKSLQLEELLAREQLEYTIEEEVAKQTIRMWLKKCLKRIRAKQQQSCSIIHSLRESQQQELSRFLNPPSIETTQPSEDTNANSQDNSMQPETSSQQQLLSPTLSDRGGSRQDAADAGKPQRKFGQWRLPSAPKPISHSVSSVNLRLGGRTTMKSVVCKMNPMTDAASCGSEVKKWWTRQLTVESDESGDDLLDI

SEQ ID NO: 2 - DNA, 6952 bp, Homo sapiens
  CDS (230)...(5443)
ggcacgagcgagcgtgagccgcggccccagccgggccgagcgcgctgcctgagctgagccgccgtaggtgaggggcccgcgtccccgcccgccctgggcgccgcgcctggcactgatcctgccggtcgcccactgtcgccgccgccgccgcccgcgggcaccatgacagctctgagcgctggggttacagactgtggttttgtgcttgctcaccaaagctaacctcagcatgctcaaaaggaagcagagttccagggtggaagcccagccagtcactgactttggtcctgatgagtctctgtcggataatgctgacatcctctggattaacaaaccatgggttcactctttgctgcgcatctgtgccatcatcagcgtcatttctgtttgtatgaatacgccaatgaccttcgagcactatcctccacttcagtatgtgaccttcactttggatacattattgatgtttctctacacggcagagatgatagcaaaaatgcacatccggggcattgtcaagggggatagttcctatgtgaaagatcgctggtgtgtttttgatggatttatggtcttttgcctttgggtttctttggtgctacaggtgtttgaaattgctgatatagttgatcagatgtcaccttggggcatgttgcggattccacggccactgattatgatccgagcattccggatttatttccgatttgaactgccaaggaccagaattacaaatattttaaagcgatcgggagaacaaatatggagtgtttccatttttctacttttctttctacttctttatggaattttaggagttcagatgtttggaacatttacttatcactgtgttgtaaatgacacaaagccagggaatgtaacctggaatagtttagctattccagacacacactgctcaccagagctagaagaaggctaccagtgcccacctggatttaaatgcatggaccttgaagatctgggacttagcaggcaagagctgggctacagtggctttaatgagataggaactagtatattcaccgtctatgaggccgcctcacaggaaggctgggtgttcctcatgtacagagcaattgacagctttccccgttggcgttcctacttctatttcatcactctcattttcttcctcgcctggcttgtgaagaacgtgtttattgctgttatcattgaaacatttgcagaaatcagagtacagtttcaacaaatgtggggatcgagaagcagcactacttcaacagccaccacccagatgtttcatgaagatgctgctggaggttggcagctggtagctgtggatgtcaacaagccccagggacgcgccccagcctgcctccagaaaatgatgcggtcatccgttttccacatgttcatcctgagcatggtgaccgtggacgtgatcgtggcggctagcaactactacaaaggagaaaacttcaggaggcagtacgacgagttctacctggcggaggtggcttttacagtactttttgatttggaagcacttctgaagatatggtgtttgggatttactggatatattagctcatctctccacaaattcgaactactactcgtaattggaactactcttcatgtatacccagatctttatcattcacaattcacgtactttcaggttctccgagtagttcggctgattaagatttcacctgcattagaagactttgtgtacaagatatttggtcctggaaaaaagcttgggagtttggttgtatttactgccagcctcttgattgttatgtcagcaattagtttgcagatgttctgctttgtcgaagaactggacagatttactacgtttccgagggcatttatgtccatgttccagatcctcacccaggaaggatgggtggacgtaatggaccaaactctaaatgctgtgggacatatgtgggcacccgtggttgccatctatttcattctctatcatctttttgccactctgatcctcctgagtttgtttgttgctgttattttggacaacttagaacttgatgaagacctaaagaagcttaaacaattaaagcaaagtgaagcaaatgcggacaccaaagaaaagctccctttacgcctgcgaatctttgaaaaatttccaaacagacctcaaatggtgaaaatctcaaagcttccttcagattttacagttcctaaaatcagggagagttttatgaagcagtttattgaccgccagcaacaggacacatgttgcctcctgagaagcctcccgaccacctcttcctcctcctgcgaccactccaaacgctcagcaattgaggacaacaaatacatcgaccaaaaacttcgcaagtctgttttcagcatcagggcaaggaaccttctggaaaaggagaccgcagtcactaaaatcttaagagcttgcacccgacagcgcatgctgagcggatcatttgaggggcagcccgcaaaggagaggtcaatcctcagcgtgcagcatcatatccgccaagagcgcaggtcactaagacatggatcaaacagccagaggatcagcaggggaaaatctcttgaaactttgactcaagatcattccaatacagtgagatatagaaatgcacaaagagaagacagtgaaataaagatgattcaggaaaaaaaggagcaagcagagatgaaaaggaaagtgcaagaagaggaactcagagagaaccacccatacttcgataagccactgttcattgtcgggcgagaacacaggttcagaaacttttgccgggtggtggtccgagcacgcttcaacgcatctaaaacagaccctgtcacaggagctgtgaaaaatacaaagtaccatcaactttatgatttgctgggattggtcacttacctggactgggtcatgatcatcgtaaccatctgctcttgcatttccatgatgtttgagtccccgtttcgaagagtcatgcatgcacctactttgcagattgctgagtatgtgtttgtgatattcatgagcattgagcttaatctgaagattatggcagatggcttatttttcactccaactgctgtcatcagggacttcggtggagtaatggacatatttatatatcttgtgagcttgatatttctttgttggatgcctcaaaatgtacctgctgaatcgggagctcagcttctaatggtccttcggtgcctgagacctctgcgcatattcaaactggtgccccagatgaggaaagttgttcgagaacttttcagcggcttcaaggaaatttttttggtctccattcttttgctgacattaatgctcgtttttgcaagctttggagttcagctttttgctggaaaactggccaagtgcaatgatcccaacattattagaagggaagattgcaatggcatattcagaattaatgtcagtgtgtcaaagaacttaaatttaaaattgaggcctggagagaaaaaacctggattttgggtgccccgtgtttgggcgaatcctcggaactttaatttcgacaatgtgggaaacgctatgctggcgttgtttgaagttctctccttgaaaggctgggtggaagtgagagatgttattattcatcgtgtggggccgatccatggaatctatattcatgtttttgtattcctgggttgcatgattggactgaccctttttgttggagtagttattgctaatttcaatgaaaacaaggggacggctttgctgaccgtcgatcagagaagatgggaagacctgaagagccgactgaagatcgcacagcctcttcatctcccgcctcgcccggataatgatggttttagagctaaaatgtatgacataacccagcatccattttttaagaggacaatcgcattactcgtcctggcccagtcggtgttgctctctgtcaagtgggacgtcgaggacccggtgaccgtacctttggcaacaatgtcagttgttttcaccttcatctttgttctggaggttaccatgaagatcatagcaatgtcgcctgctggcttctggcaaagcagaagaaaccgatacgatctcctggtgacgtcgcttggcgttgtatgggtggtgcttcactttgccctcctgaatgcatatacttacatgatgggcgcttgtgtgattgtatttaggtttttctccatctgtggaaaacatgtaacgctaaagatgctcctcttgacagtggtcgtcagcatgtacaagagcttctttatcatagtaggcatgtttctcttgctgctgtgttacgcttttgctggagttgttttatttggtactgtgaaatatggggagaatattaacaggcatgcaaatttttcttcggctggaaaagctattaccgtactgttccgaattgtcacaggtgaagactggaacaagattatgcatgactgtatggttcagcctccgttttgtactccagatgaatttacatactgggcaacagactgtggaaattatgctggggcacttatgtatttctgttcattttatgtcatcattgcctacatcatgctaaatctgcttgtagccataattgtggagaatttctccttgttttattccactgaggaggaccagcttttaagttacaatgatcttcgccactttcaaatcatatggaacatggtggatgataaaagagagggggtgatccccacgttccgcgtcaagttcctgctgcggctactgcgtgggaggctggaggtggacctggacaaggacaagctcctgtttaagcacatgtgctacgaaatggagaggctccacaatggcggcgacgtcaccttccatgatgtcctgagcatgctttcataccggtccgtggacatccggaagagcttgcagctggaggaactcctggcgagggagcagctggagtacaccatagaggaggaggtggccaagcagaccatccgcatgtggctcaagaagtgcctgaagcgcatcagagctaaacagcagcagtcgtgcagtatcatccacagcctgagagagagtcagcagcaagagctgagccggtttctgaacccgcccagcatcgagaccacccagcccagtgaggacacgaatgccaacagtcaggacaacagcatgcaacctgagacaagcagccagcagcagctcctgagccccacgctgtcggatagaggaggaagtcggcaagatgcagccgacgcagggaaaccccagaggaaatttgggcagtggcgtctgccatcagccccaaaaccaataagccattcagtgtcctcagtcaacttacggttaggaggaaggacaaccatgaaatctgtcgtgtgcaaaatgaaccccatgactgacgcggcttcctgcggttctgaagttaagaagtggtggacccggcagctgactgtggagagcgacgaaagtggggatgaccttctggatatttaggtggatgtcaatgtagatgaatttctagggtggaaaccgttttctaataatgtccttgattgtccagtgagcaatctgtaattgactataactgaattccagcttgtcacaagatgtttataaattgattttcatcctgccacagaaaggcataagctgcagtatgatgggttactatcaatcattgctcaaaaaaatttttgtataatgacagtactgataatattagaaatgataccgcaagcaaatgtatatcattaaaaatgtcatatattctgtctgcgtaaactaagtataattcatattgctctaatagtattatcaccgccccsmaaagagtgctaagcccaaagtggctgatatttagggtacaggggttatagctttagttcacatctttcccatttccactagaaattttctcgagagaatttattatttatgattgatctgaaaaggtcagcactgaacttatgctaaatgatagtagttttacaaactacagattctgaattttaaaaagtatcttctttttctcgtgttatatttttaaatatacacaagacatttggtgaccagaacaagttgatttctgtcctcagttatgttaatgaaactgttgcctccttctaagaaaattgtgtgtgcaagcaccaggcaaagaaatggactcaggatgcttagcggtttaaaacaaacctgtagataaatcacttgagtgacatagttgcgcaaagatgttaagtttcttaagaaaccttttaataactgagtttagcaaaaagaataaaactatatagctcaatttatttaaaaaaatctttttgcatgtgtgatgttatcattggcttcatttcttacccaaggtatgtctgttttgccataaatcagcagagtcatttcattctgggtgatcctgacacaccattgctatgttagatttgaaatgacatctctgttaaaagaatcttctatggaaataatggtgccctgcaaaatctycctctgaactcacaggttagggatcacacaacttacttaatcgttttttgtttttgtttttttyccttatatgtcaatcggcccatgtcctccgggaaaattagaaaagcaaaatgattacaaagtgctgttagatttcttgtgctgggccagccaagtagaagtggacttgacttggacctttaactattttattacagattggacatttgctgttcagatgttttttaacagagggattatctcagaatcctgtgacctccaggttgttttataatctatttttctctatttaacattcctcagatagataggcaaataggacattccttctgtgtcacagaagtatcgtggtagtggcagtctacagtttatatgattcattgtaactatgagataaagaacaaccagtcatgtggccaaaaggattagatttgattggatgttcacttggagtttactttttgtacatacaagataaaataaatattggatttgtaaaat

SEQ ID NO: 3 - protein, 1738 aa, Homo sapiens; identical to SEQ ID NO: 1

(19) United States
(12) Patent Application Publication
(10) Pub. No.: US 2003/0078403 A1
(43) Pub. Date: Apr. 24, 2003

(54) BION1 is a novel ion channel

(76) Inventors: Leonard Kaczmarek, Guilford, CT (US); Michael Tang, New Haven, CT (US); Manjunath Nimmakayalu, New Haven, CT (US); Patricia Bray-Ward, Madison, CT (US)

Correspondence Address: BANNER & WITCOFF, 1001 G STREET N W, SUITE 1100, WASHINGTON, DC, 20001, US

(21) Appl. No.: 10/178,791

(22) Filed: Jun. 25, 2002

Related U.S. Application Data
(60) Provisional application No. 60/300,101, filed on Jun. 25, 2001.

(51) Int. Cl. C07H 21/04; C12Q 1/68

(52) U.S. Cl. 536/23.2; 435/6

(57) ABSTRACT

Polymorphisms are identified in a human gene which encodes a voltage-gated ion channel. The gene maps to the same portion of human chromosome 13q22 to which genes for schizophrenia susceptibility, Bipolar Disorder, and Panic Disorder Syndrome have been mapped. The polymorphisms are used to follow inheritance of the susceptibilities within families. They are also used to identify affected probands. A complete mRNA sequence and genomic structure provide insights into function and relationship to other ion channels.

This application claims priority from Provisional Application Serial No. 60/300,101 filed Jun. 25, 2001, the contents of which are hereby incorporated by reference.

### TECHNICAL FIELD OF THE INVENTION

This invention is related to ion channels expressed in brain tissue. In particular it is related to ion channels which are associated with susceptibility to psychiatric disorders.

### BACKGROUND OF THE INVENTION

It has long been known that the flow of ions in and out of cells in the nervous system is crucial for their activity, and that specific ion channels are largely responsible for this movement. Ion channels are generally gated (with the exception of the K⁺ leak channels), either by ligand binding or by voltage. Ligand binding ion channels convert extracellular chemical signals into electrical signals, while voltage gated ion channels, particularly Na⁺ channels, play a key role in action potential propagation. The voltage gated Ca⁺⁺ channel provides the only known means of converting electrical signals into chemical signals. We describe here the isolation and characterization of BION1 (Brain Ion 1), a novel voltage gated channel protein of unknown ion specificity. Various lines of evidence are presented that link the gene encoding this protein with a schizophrenia susceptibility locus on chromosome 13q32.

Schizophrenia is a serious disorder characterized by severe psychotic symptoms, and is fairly common, affecting ˜1% of the general population. The illness often develops in young adults who were previously normal, and is characterized by a constellation of symptoms including hallucinations and delusions (psychotic symptoms) and symptoms such as severely inappropriate emotional responses, disordered thinking and concentration, erratic behavior, as well as social and occupational deterioration (Andreasen, 1995). Family, twin and adoption studies indicate that schizophrenia is primarily (71%) genetic. The genetic component of schizophrenia is complex, polygenic, and involves epistatic interaction between loci. Mapping studies reveal that this genetic component can be mapped to several distinct genetic loci, including SCZD7 on 13q32 (Blouin et al. 1998, Pulver et al. 1998, Shaw et al. 1998, Brzustowicz et al. 1999, Brzustowicz et al. 2000).

Recently, a genetic linkage between 13q32 and a second syndrome, Panic Disorder Syndrome (Weissman et al. 2000) has been established. Panic Disorder (anxiety neurosis) is also genetic, with a prevalence of 1-3%, and is symptomatically represented by recurrent episodes of sudden apprehension and associated autonomic symptoms involving the cardio-respiratory system, nervous system, and gastrointestinal system. Panic Disorder Syndrome occurs in a subset of Panic Disorder patients, and is additionally associated with an increased rate of mitral valve prolapse, bladder/kidney problems, serious headaches and/or thyroid problems. When these additional symptoms are scored, a high LOD (likelihood of linkage) score was obtained with one marker, D13S779, which is also at 13q32. Interestingly, when only Panic Disorder was considered, there was no significant LOD with this marker, suggesting that panic disorder is also polygenic (Knowles et al. 1998). While multiple case reports of familial association of schizophrenia and Panic Disorder exist in the literature, statistical association of the two disorders has not been performed. Bipolar Disorder, another serious psychiatric disorder, has also been found to be genetically linked to a locus at or near chromosome 13q32.

There is a need in the art for additional tools for diagnosing and studying psychiatric disorders, including schizophrenia, Bipolar Disorder, and Panic Disorder Syndrome.

### SUMMARY OF THE INVENTION

It is an object of the present invention to provide an isolated and purified polynucleotide which encodes human BION1.

It is another object of the present invention to provide a method of determining susceptibility to Schizophrenia, Bipolar Disorder, or Panic Disorder Syndrome.

It is an object of the present invention to provide an isolated and purified polynucleotide comprising at least 18 contiguous nucleotides of a human BION1 coding sequence.

Another object of the invention is to provide an isolated and purified human BION1 protein.

Another object of the invention is to provide an isolated and purified human BION1 polypeptide comprising at least 6 amino acids of a human BION1 protein.

Another object of the invention is to provide a method of producing human BION1.

Another object of the invention is to provide a method of screening test substances for candidates useful in treating Schizophrenia, Bipolar Disorder, or Panic Disorder Syndrome.

These and other objects of the invention are achieved by providing one or more of the embodiments shown below. In one embodiment of the invention an isolated and purified polynucleotide which encodes human BION1 is provided. The polynucleotide encodes an amino acid sequence as shown in SEQ ID NO:1 or a polymorphic variant found in a schizophrenic, Bipolar Disorder, or Panic Disorder Syndrome patient.

Another aspect of the invention is an isolated and purified polynucleotide comprising at least 18 contiguous nucleotides of a human BION1 coding sequence. The polynucleotide comprises at least one codon identified in Table 1, which codon is for an amino acid found in human BION1 proteins at the corresponding position.

Still another embodiment provided by the present invention is an isolated and purified human BION1 protein. The protein comprises an amino acid sequence as shown in SEQ ID NO:1 or a polymorphic variant found in Schizophrenia, Bipolar Disorder, or Panic Disorder Syndrome patients.

According to yet another embodiment of the invention, an isolated and purified human BION1 polypeptide comprising at least 6 amino acids of a human BION1 protein is provided. The polypeptide comprises an amino acid sequence found in humans but not in rats as identified in Table 1.

According to another embodiment of the invention an isolated and purified polynucleotide of a human BION1 gene is provided. The polynucleotide comprises a polymorphic nucleotide identified in Table 2 or Table 3.

Even another embodiment provided by the present invention is a method of producing human BION1. A host cell comprising a vector which encodes a human BION1 protein is cultured under conditions for expression of BION1 from the vector. BION1 protein is then collected from the cultured host cells or culture medium.

According to another aspect of the invention a method is provided for screening test substances for candidates useful in treating Schizophrenia, Bipolar Disorder, or Panic Disorder Syndrome. A human BION1 protein is contacted with a test substance. Test substances are determined which bind to the human BION1 protein. A test substance is identified as a candidate drug useful for treating Schizophrenia, Bipolar Disorder, or Panic Disorder Syndrome if it binds to human BION1.

Thus the present invention provides diagnostic and drug development tools for addressing psychiatric disorders for which genetic and pharmacologic interventions are needed.

### BRIEF DESCRIPTION OF THE DRAWINGS

FIG. 1a: cDNA sequence of human BION1. The complete coding sequence is underlined, running from nucleotide 230 to 5443..

FIG. 1b: Protein sequence of human BION1. Protein sequence of human BION1. The positions of the transmembrane domains and loops are shown underlined. The residues in the loop thought to be responsible for ion selectivity (EEKE) are in red bold face.

FIG. 2a: The predicted secondary structure of BION1, showing its predicted topology within the cell membrane. Additionally, the predicted pore structure of Bion1, Naᵥ, and Caᵥ channels is shown

FIG. 2b: Predicted Secondary structure of BION1, showing its predicted topology within the cell membrane. Additionally, the predicted pore structure of Bion1, Naᵥ, and Caᵥ channels is shown.

FIG. 3a: Amino acid residues thought to be crucial in determining ion selectivity demonstrate the relationship between BION1, Na⁺ channels (type II from brain), and Ca⁺⁺ channels (L-type from heart). Note the conserved DEKA in Na⁺ channels and EEEE in Ca⁺⁺ channels is replaced by the novel combination EEKE.

FIG. 3b: Phylogenetic tree relating different members of the voltage-gated ion channel superfamily. Alignment of the amino acid sequences of S4 regions of different members of the voltage-gated ion channels was generated using CLUSTALW.

FIG. 4a: Northern blot of rat poly(A)⁺ RNA isolated from a variety of tissues probed with Bion1. Highest expression is in brain and pancreas (not shown), although significant expression is also seen in heart, kidney, small intestine and placenta.

FIG. 4b: Northern blot of rat poly(A)⁺ RNA isolated from different parts of the brain probed with Bion1. Densitometry readings relative to thalamic expression are given.

FIG. 5a: In situ hybridization of BION1 to rat brain sections. Note the strong signal in the hippocampus (hip) in panel A.

FIG. 5b: Emulsion autoradiogram of in situ hybridization of BION1 to rat brain sections. Note strong signal over the neuronal layer of the hippocampus (panel A).

FIG. 6a: Fluorescent in situ hybridization with BION1 showing hybridization to human chromosome 13q22. DAPI DNA staining in blue and the FISH signal in red.

FIG. 6b: The same karyotype as in FIG. 6a has been alu-banded in green.

FIG. 7: BION1 gene structure. Protein coding sequence is shown in shaded.

FIG. 8: BION1 polymorphism analysis. The positions of all polymorphisms found are marked with an asterisk. Intronic polymorphisms are indicated by minus signs in the name (e.g., T(−221)C, indicating that it is 221 nt from the intron/exon boundary).

### DETAILED DESCRIPTION OF THE PREFERRED EMBODIMENTS

We have determined the sequence of the human homolog of the rat BION1 gene, a voltage-gated ion channel with novel ion specificity sequence. We have localized this gene to human chromosome 13q32, where a schizophrenia susceptibility gene (SCZD7), Bipolar Disorder locus, and Panic Disorder Syndrome locus map genetically. Moreover, the closest marker to these genetic loci, D13S779, is contained within a YAC clone that also contains BION1. We have shown by Northern blot analysis that rat BION1 RNA is expressed in the brain, consistent with a neuroactive protein. Interestingly, it is also expressed moderately in the heart; the subset of Panic Disorder patients that comprise the Panic Disorder Syndrome exhibit a high incidence of mitral valve prolapse. In situ hybridization to rat brain sections confirmed that staining was chiefly confined to neuronal cell types. We have identified the genomic structure of BION1 for all 42 exons. Sequencing of 13 exons, which comprises 27% of the coding region has revealed 3 exonic, 4 intronic, and 4 5′UTR polymorphisms.

An isolated and purified polynucleotide according to the present invention typically encodes human BION1, which has an amino acid sequence as shown in SEQ ID NO:1. Polymorphic variants found in a schizophrenic, Bipolar Disorder, or Panic Disorder Syndrome patient are also included within the polynucleotides contemplated. The polynucleotides can be cDNA, genomic DNA, RNA or other forms of polynucleotides. The polynucleotide can be in an expression vector or a non-expression vector, such as a YAC clone, a BAC clone, a P1 clone, and the like. Preferably a genomic clone also contains marker D135779, which has been found to be closely linked to Panic Disorder Syndrome and schizophrenia. Preferably the polynucleotide includes at least 12, 15, 18, 20, 22, 25, 30, 35, 50, 75, 100, 250, 500, or 1000 nucleotides. More preferably the entire amino acid-coding sequence shown in SEQ ID NO:2 is contained within the polynucleotide. In another embodiment the complete cDNA sequence shown in SEQ ID NO:2 is contained within the polynucleotide. If the polynucleotide is an RNA molecule one preferred embodiment is a 6.2 kb transcript.

Polynucleotides according to the invention contain either the wild type sequence as shown in SEQ ID NO:2 or polymorphisms, such as those shown in Table 2 or Table 3. Other polymorphisms can be readily identified using the method shown below in the examples. Isolated and purified polynucleotides according to the present invention are separated from the sequences to which they are adjacent in the human genome. Thus such isolated polynucleotides comprise less than a full chromosome or other genomic element. Moreover, purified polynucleotides are not present in a mixture of total genomic DNA or a library comprising total genomic DNA or cDNA. Purified polynucleotides have been separated from other sequences, usually by a process such as cloning, hybridization, or amplification such that a population of polynucleotide molecules is predominantly (>50%) the type of molecule which contains the polynucleotide containing BION1 sequences.

Polynucleotides according to the present invention are conveniently maintained, propagated, and expressed in a vector. The vector can be any useful vector known in the art, whether bacterial or eukaryotic, viral or plasmid. Vectors can be maintained and grown in host cells which are suitable for replication of the particular vector chosen. Appropriate pairs of vectors and host cells are well known in the art. Such host cells carrying BION1 coding sequences can be used to prepare quantities of BION1 protein. Host cells can be grown under suitable growth and expression conditions. BION1 will typically be extracted from the cells, but under certain conditions and with certain genetic constructs, may be isolatable from the culture medium.

Susceptibility to Schizophrenia, Bipolar Disorder, or Panic Disorder Syndrome can be determined according to one aspect of the invention by tracking a polymorphism identified in a proband through other family members. The proband is an affected individual, having either Schizophrenia, Bipolar Disorder, or Panic Disorder Syndrome. A nucleotide at a defined location in a BION1 gene of a human is determined to identify whether the test individual contains the polymorphism carried by the proband. The polymorphic nucleotide can be one of the polymorphisms indicated in Tables 2 and 3 or can be a different nucleotide in the same codon, or a different nucleotide in a different codon. The nucleotide at the defined location can be determined by any means known in the art, including but not limited to sequencing, restriction enzyme digestion, allele-specific ligation, allele-specific amplification. The nucleotide at the defined location of the human is compared to that of the affected family member (proband) having a polymorphism at the nucleotide. Such comparison can be by any means known in the art, such as by side-by-side testing, or by serial testing at two different times, with the same or a different technique. The testing and/or comparison can be mannually accomplished or can be done by a machine or computer implemented technique. The human being tested is identified as susceptible to schizophrenia or panic disorder if the determined nucleotide contains the polymorphism found in the affected family member. As is well known in the art, susceptibility does not indicate that there is a 100% probability that the individual will develop the disease symptoms. Other genetic factors and environmental factors are believed to be involved in determining disease elaboration.

Another method of determining susceptibility to Schizophrenia, Bipolar Disorder, or Panic Disorder Syndrome requires knowledge of no other affected family member. Thus the tested patient may be the first in a family to be affected or may have no knowledge of prior affected members, or prior affected family members may not be available for sample donation. A nucleotide at a defined location in a BION1 gene of a human is determined. If the determined nucleotide is a polymorphism identified in Table 2 or Table 3, the human is identified as susceptible to Panic Disorder Syndrome, Bipolar Disorder, or Schizophrenia. As the collection of identified polymorphisms grows, this knowledge can be used to identify susceptibility in others. Thus the list of Table 2 or Table 3 will become more extensive as additional knowledge accumulates regarding other families and other polymorphisms. The expanded list of polymorphisms can be used, like Table 2 or Table 3 as provided herein. Thus Table 2 and Table 3 represent any set of polymorphisms in BION1 previously identified at the time of performing the method.

Probes and primers for BION1 are also provided which are useful for performing diagnoses according to the invention. Polynucleotides for use as primers or probes typically comprise at least 18 contiguous nucleotides of a human BION1 coding sequence, however, primers and probes of at least 10, 12, 14, 16, 20, 22, or 25 nucleotides may be useful, as well. Polynucleotides encoding only a portion of BION1 can also be fused to other genes to form fusion genes encoding fusion proteins. Preferably the polynucleotide comprises at least one codon identified in Table 1, which codon is for an amino acid found in human BION1 proteins at the corresponding position. More preferably the polynucleotide contains no codons identified in Table 1 as being a rat BION1 codon.

Human BION1 protein according to the invention is useful inter alia for screening for potential therapeutic agents. Typically such protein is isolated and purified away from whole human cells or recombinant cells, as well as away from other human proteins and possibly also other proteins from a recombinant BION1-producing cell. The BION1 proteins according to the invention typically comprise an amino acid sequence as shown in SEQ ID NO:1. Alternatively the sequence can be that of a polymorphic variant found in Schizophrenia, Bipolar Disorder, or Panic Disorder Syndrome patients. More preferably it can be the sequence of a polymorphic variant disclosed in Tables 2 or 3.

BION1 polypeptides comprise at least 6 amino acids of a human BION1 protein, i.e., enought to form an epitope which can be used to generate antibodies or immune cells which are specific for BION1. The polypeptides can comprise at least 10, 15, 20, 25, 30, 50, or even 75 contiguous amino acids of the BION1 protein. Preferably the polypeptide comprises an amino acid sequence found in humans but not in rats as identified in Table 1. Among other uses, the antibodies to BION1 can be used in binding assays to test substances which may be useful therapeutically. The antibodeies, whether polyclonal or monoclonal, can be used to identify bound or unbound BION1 in an assay or as a competitor molecule for BION1 binding.

Test substances can be screened for candidates useful in treating Schizophrenia, Bipolar Disorder, or Panic Disorder Syndrome by using the human BION1 protein. It is contacted with a test substance, preferably with a library of test substances serially or simultaneously in separate reaction mixtures. Binding of a test substance to the human BION1 protein is determined. The BION1 protein may be wild-type or polymorphic, including but not limited to one of the polymorphisms disclosed in Tables 2 and 3. Any binding assay known in the art can be used. In some binding assays one of the binding partners is immobilized on a solid support. In other binding assays one binding partner is labeled. In some binding assays competition with a known binder is used as an indicator of binding activity. Those of skill in the art will readily understand how to set up convenient binding assays. A test substance is identified as a candidate drug useful for treating Schizophrenia, Bipolar Disorder, or Panic Disorder Syndrome if it binds to human BION1.

The above disclosure generally describes the present invention. A more complete understanding can be obtained by reference to the following specific examples which are provided herein for purposes of illustration only, and are not intended to limit the scope of the invention.

### EXAMPLE 1

Identification of C. elegans Homologs of Mammalian Voltage-Gated Ion Channels.

Sequences were identified within the C. elegans cosmids c27f2.3 (U40419) and c11d2.6 (AF045640) in a search of the C. elegans genomic database for sequence fragments that resembled voltage gated sodium (NaV) and calcium (CaV) ion channels. These putative, novel genes were approximately 26% identical, 45% conserved (e-value 2e-48) to human voltage-dependent L-type Ca channel (al subunit).

### EXAMPLE 2

Cloning of Human BION1 cDNA.

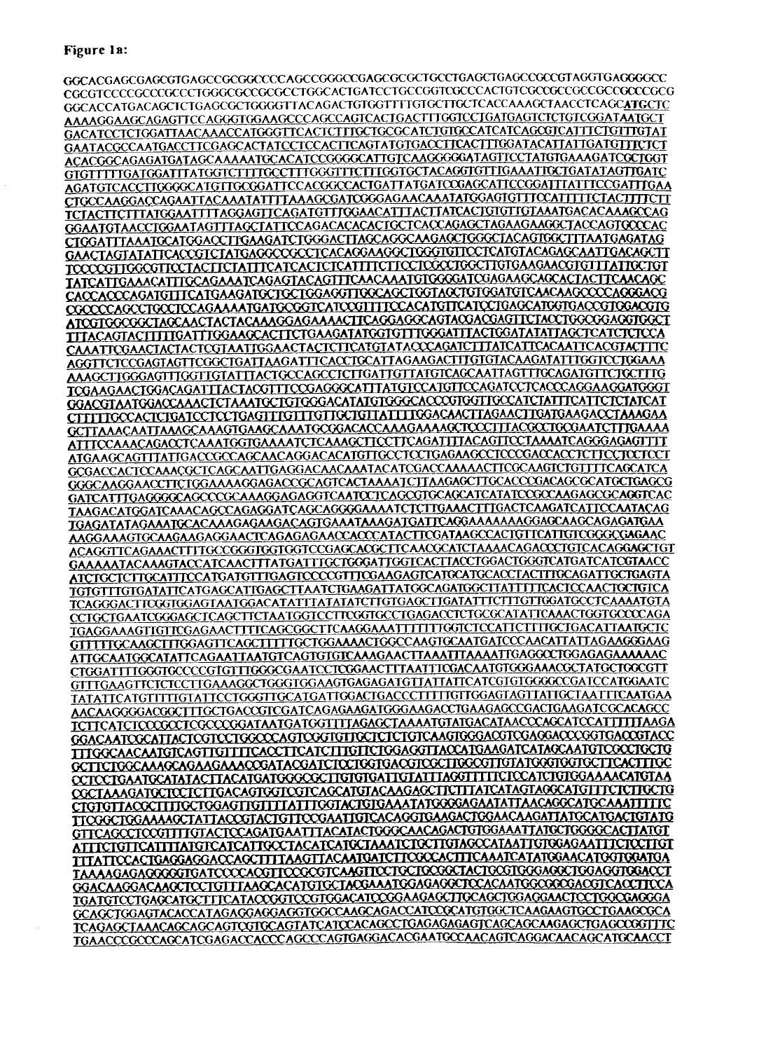

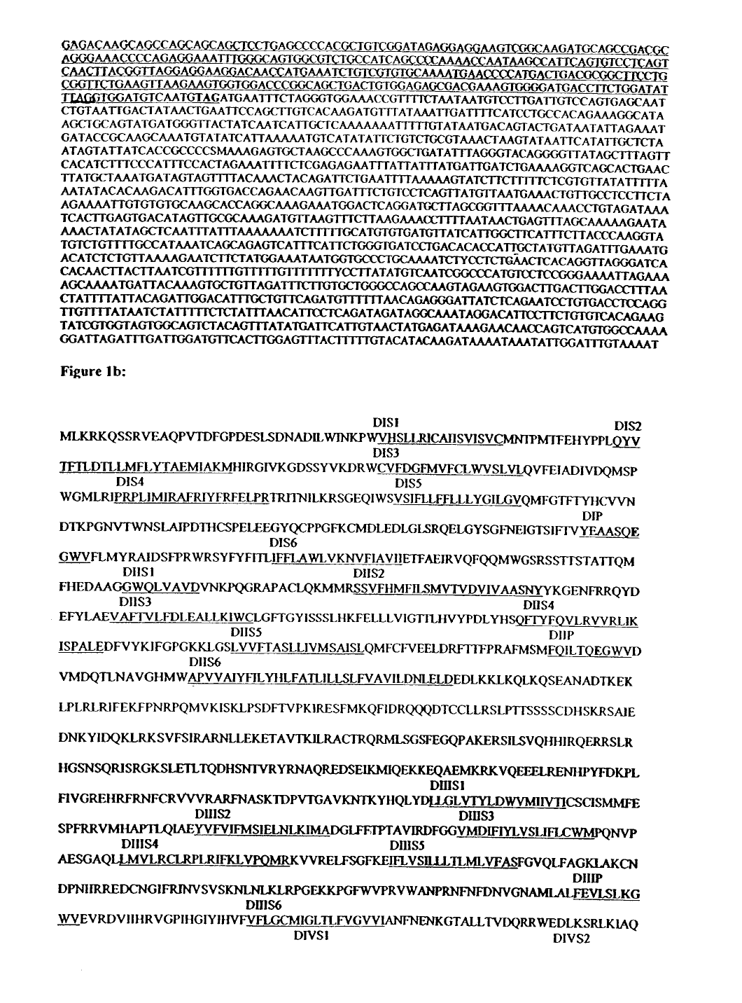

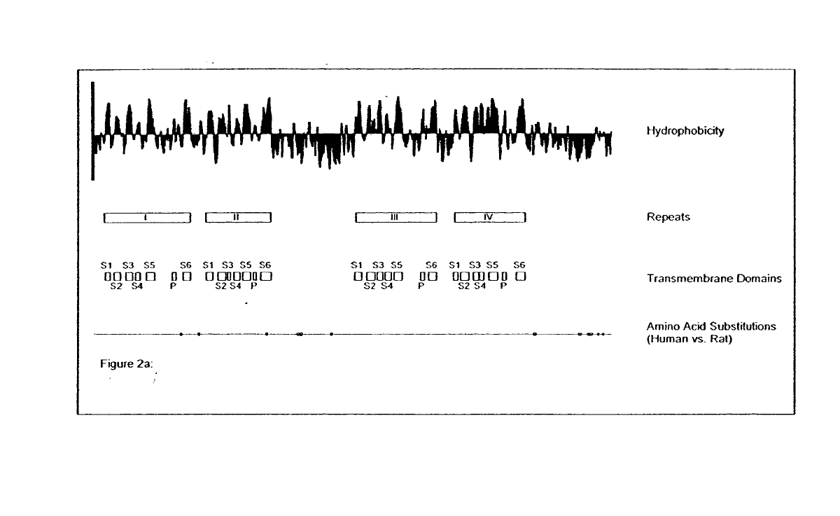

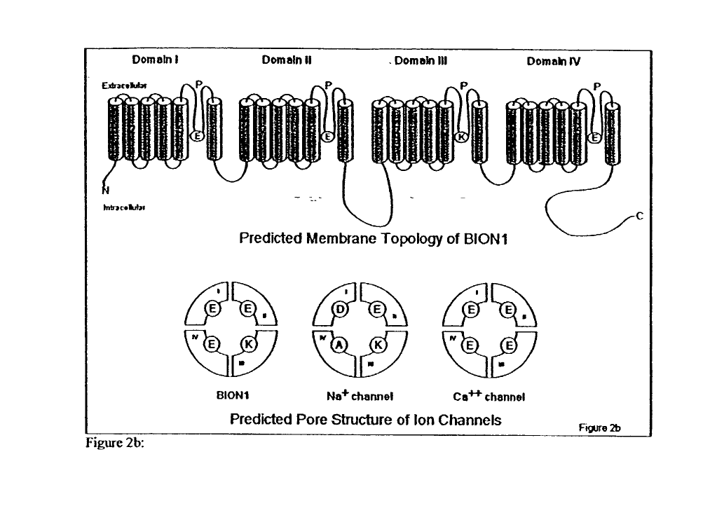

These C. elegans sequences displaying similarity to the ion channels were used to identify a homologous human GenBank EST (#590575, derived from a human pancreatic cell line cDNA library.) This was sequenced and found to contain an open reading frame (ORF) encoding part of the human homolog of the C. elegans genes. Further screening of a pancreatic cDNA library led to the isolation of a full-length human cDNA clone (BION1). The complete human cDNA sequence of the human BION1 gene (SEQ ID NO:2) and its conceptual translation product (SEQ ID NO:1) are shown in FIG. 1. The predicted human and rat protein sequences contain 1738 amino acids, and are highly conserved, showing only 24 cross-species amino acid substitutions (1.4%), 12 of which are conservative. The predicted BION1 protein is comprised of four domains (I-IV), each containing six putative membrane-spanning regions (S1-6) and a pore loop (P) between S5 and S6 (FIG. 2a). The predicted topology of the BION1 protein is shown diagrammatically in FIG. 2b.

TABLE 1
Amino acid substitutions between human and rat BION1 proteins.

| Position of first | Amino acid | Amino acid | |
|---|---|---|---|
| nucleotide in codon | in human | in rat | Conservative? |
| 1058 | Ala | Ser | Conservative |
| 1241 | Ser | Thr | Conservative |
| 1949 | Val | Leu | Conservative |
| 2069 | Val | Leu | Conservative |
| 2261 | Leu | Phe | |
| 2267 | Ser | Ile | |
| 2276 | Thr | Ser | Conservative |
| 2300 | His | Asn | Conservative |
| 2303 | Ser | Pro | |
| 2312 | Ser | Pro | |
| 2315 | Ala | Thr | |
| 2318 | Ile | Val | Conservative |
| 2909 | Ile | Leu | Conservative |
| 4687 | Lys | Ser | |
| 4670 | Arg | ? | |
| 5117 | Asn | His | Conservative |
| 5120 | Ser | Asn | Conservative |
| 5123 | Met | Thr | Conservative |
| 5135 | Thr | Ser | Conservative |
| 5207 | Ala | Thr | |
| 5228 | Phe | Ile | |
| 5297 | Leu | Phe | |
| 5348 | Thr | Pro | |
| 5354 | Ala | Thr | |

### EXAMPLE 3

Cloning of Rat BION1 cDNA.

The human BION1 cDNA was used to screen a rat brain cDNA library in order to isolate a full-length rat BION1 channel cDNA. At the amino-acid level, this protein exhibited 98.6% identity to the existing human sequence. Recently, another group has reported cloning and sequencing the rat BION1 gene (AF078779) (Lee et al. 1999.)

### EXAMPLE 4

BION1 Protein Sequence is Divergent from Other Voltage-Gated Ion Channels.

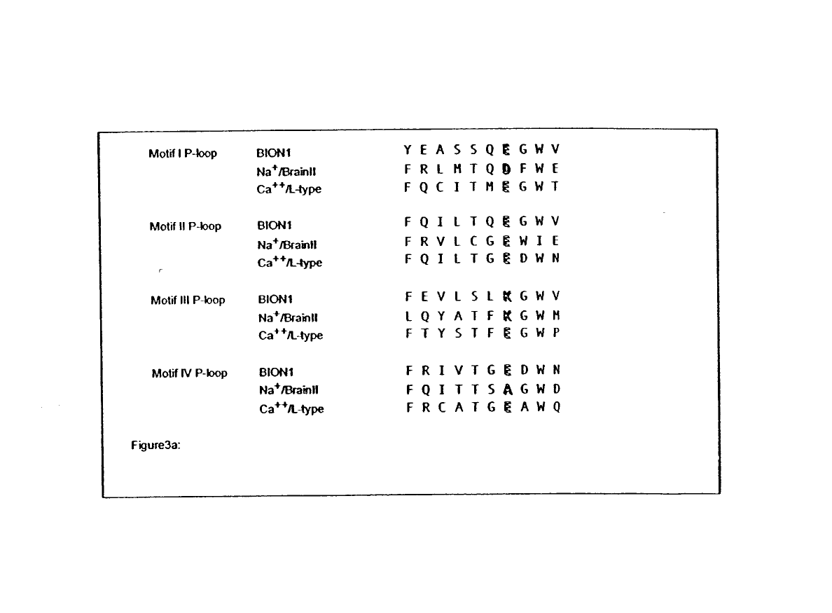

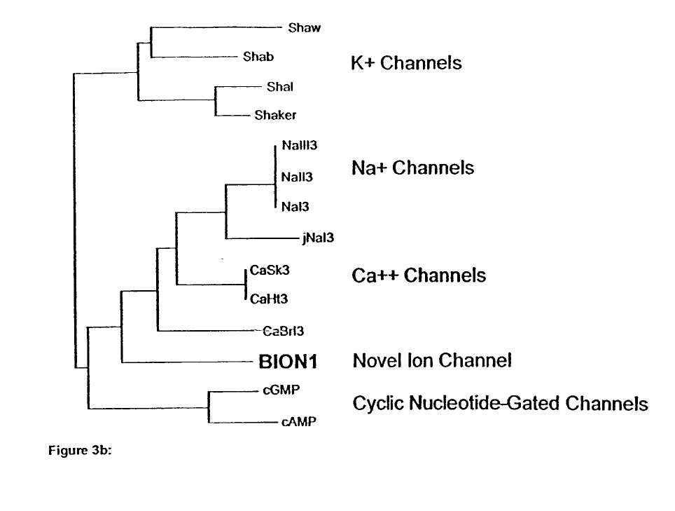

Sequence alignments of BION1 with other voltage gated ion channels indicate significant divergence. Amino-acid residues thought crucial for determining ion selectivity are in a novel arrangement. BION1 has EEKE selectivity amino acids in its pore loop region, in contrast to most mammalian Ca_{V} channels with EEEE at these positions, and Na_{V} with DEKA (FIG. 3a). Cladistic analysis of BION1 and its C. elegans homologs suggests that these comprise a distinct family of channels that diverged from Ca_{V} and Na_{V} before the duplication that gave rise to these two more closely related families (FIG. 3b). BION1 family members have a reduced number of positively charged amino-acid residues in the S4 regions, which are believed to act as part of the voltage-sensing apparatus in other families of voltage-dependent ion channels. This reduction in number of charges is particularly pronounced in domain IV, and is suggestive that the activation of this channel may be relatively slow compared to Ca_{V} and Na_{V}. BION1 channels have a divergent domain III-IV linker, missing the amino-acid sequence IFM, which has been shown to be important in mammalian Na_{V} channel inactivation. This suggests that BION1 channel inactivation may be slow compared to Na_{V}.

### EXAMPLE 5

Expression of BION1.

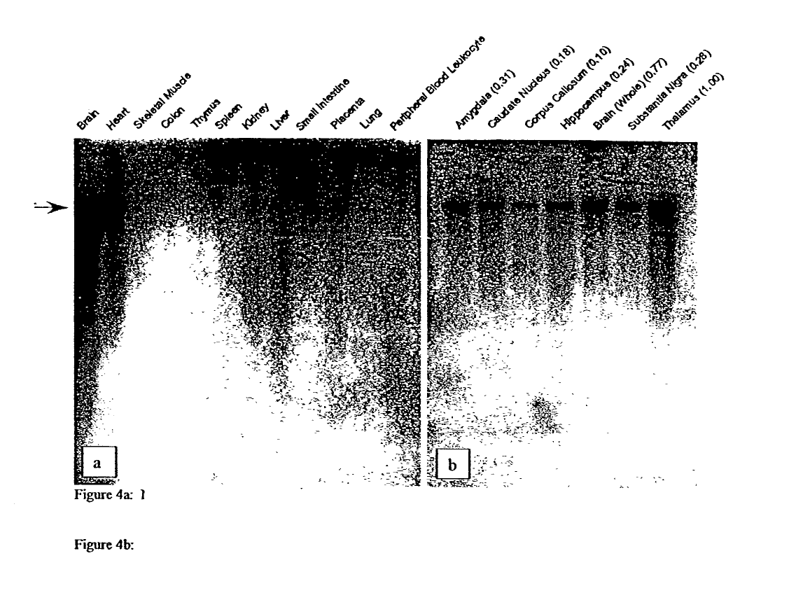

Northern analyses were performed using the rat BION1 gene. BION1 was found to be expressed abundantly in rat brain, spinal cord, and pancreas as a ˜6.2 kb transcript (FIG. 4a). Significant expression is also seen in heart, kidney, small intestine and placenta. The expression in heart is of significance given the mitral valve prolapse phenotype of Panic Disorder Syndrome.

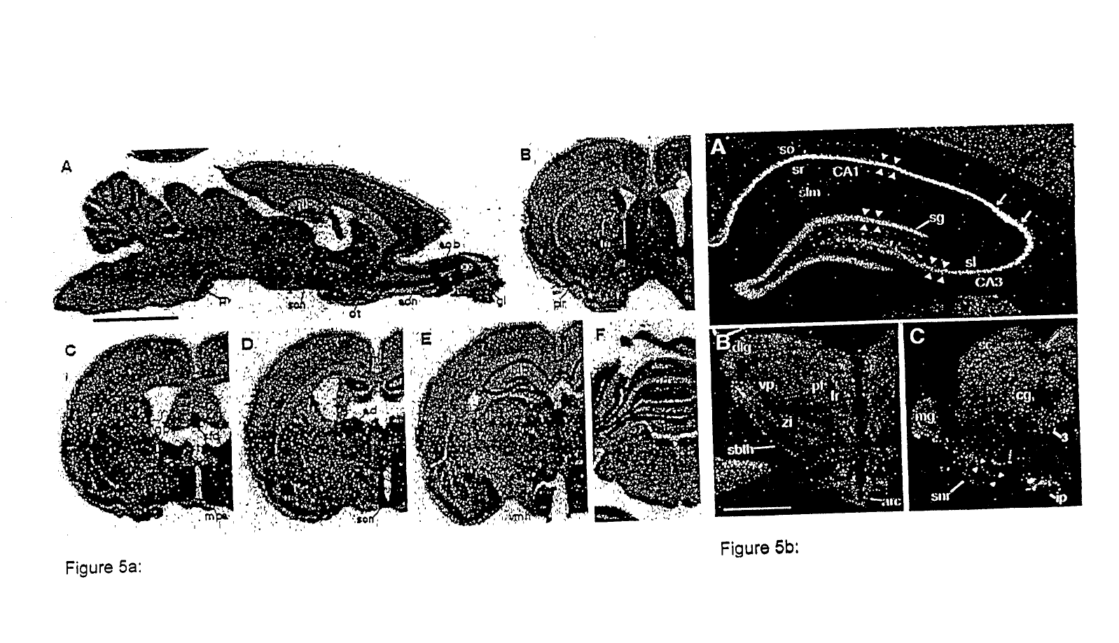

In the brain, BION1 is expressed most highly in the thalamus, but is also expressed in other regions of the brain (FIG. 4b). This distribution has been confirmed by RT-PCR. In situ hybridization using BION1 cRNA shows primarily neuronal staining, with strong hybridization to the olfactory bulb, piriform cortex, hippocampal neuronal layers, suprachiasmatic nucleus, medial habenular nucleus, locus coeruleus, and subformical organ, and at low levels throughout the CNS (FIG. 5).

### EXAMPLE 6

Chromosomal Mapping of BION1.

A BAC clone (2294N13) was shown to contain BION1 by PCR analysis and sequencing using BION1 primers. FISH analysis of the BION1 BAC clone in the Ward laboratory (Yale University) localized the human gene to human chromosomal band 13q32 (FIG. 6).

A well-defined schizophrenia susceptibility locus (SCZD7) is located on 13q32 (Lin et al. 1995, Blouin et al. 1998). The genetic linkage marker with the highest LOD score was CHLC.ATA26D07 (D13S779)(Blouin et al. 1998). A second neuropsychiatric disorder, Panic Disorder, was also linked to the D13S779 marker (Weissman et al. 2000).

A YAC contig covering the 13q32 region including and surrounding the D13S779 marker was developed in the Ward laboratory and 2 YACs from the contig were demonstrated to contain the BION1 gene by PCR analysis using BION1 cDNA primers. The 2 YACs (968g12 and 761a1) share a single STS marker, D13S919, which is immediately adjacent to D13S779 on the MIT genetic linkage map. D13S779 is found only in YAC 761al. Thus BION1 physically maps to the same YAC as D13S779, the genetic marker with the highest LOD score with the 13q32 schizophrenia susceptibility locus.

### EXAMPLE 7

Genomic Structure of BION1.

In order to determine the genomic structure of BION1, the BION1 BAC clone was sequenced in its entirety and additional overlapping BACs were identified, both by screening various BAC libraries with the available ESTs, and by database analysis. Genomic sequence of a second BION1-containing BAC, RP11-45P5, was recently completed as part of the Human Genome Project (Birren et al., unpublished). Together, these BAC sequences have allowed us to completely define the genomic structure of BION1. The BION1 gene is composed of 42 exons (FIG. 7). The 3′ portion of this gene (corresponding to bp 1855-6942 of the BION1 cDNA) spans 175 kb of genomic DNA; sequencing of the 5′ introns is as yet incomplete. However, by analysis of sequence of the two BAC clones, and by genomic PCR and sequencing, intron-exon boundaries and intronic sequences flanking individual exons have been obtained for all 42 exons.

### EXAMPLE 8

Evidence Linking BION1 to SCZD7.

Genetic co-localization of BION1 and SCZD7, together with congruent tissue distribution and predicted function of the BION1 protein, constitutes strong but indirect evidence that the BION1 gene is the 13q32 schizophrenia susceptibility gene SCZD7. In order to validate this hypothesis, mutational analysis of BION1 was undertaken in cohorts of schizophrenia and Panic Disorder Syndrome patients.

Approach to Identify Mutations in BION1.

Given the polygenic nature of schizophrenia susceptibility, sequencing of the BION1 exons from the genomic DNA of large numbers of unrelated patient probands (48 Panic Disorder and 48 Schizophrenic patient probands) was undertaken in an attempt to identify mutations.

Panic Disorder Patient Selection.

We were fortunate to have access to the same cohort that Weissman et al. (2000) used to map Panic Disorder Syndrome to 13q32. Families were initially accepted into this study if at least 3 relatives appeared affected by panic disorder. A detailed diagnosis and pedigree analysis were then performed, and the data blinded to name and family. Three senior clinical investigators independently diagnosed each individual, and any case that had a discrepant diagnosis among the physicians was flagged for additional data collection and review. Since these samples were closely linked to 13q32, it was expected that all of these patients would be defective at 13q32.

Schizophrenia Patient Selection.

Genomic DNA from DSM-III-R criteria (Diagnostic and Statistical Manual of Mental Disorders) schizophrenic patient probands was obtained from the NIMH Schizophrenia Genetics Initiative collection. Samples are included in this collection if several conditions are met. Firstly, at least two affected individuals must be biologically related as first-degree relatives diagnosed with DSM-III-R schizophrenia (SZ) or schizoaffective disorder depressive type (SADD). If this condition is met, a pedigree is established and extended. Two senior psychiatrists or clinical psychologists separately and independently assess data from clinical interviews and family history to arrive at a diagnosis. In the case of a disagreement, a third senior psychiatrist or clinical psychologist reviewed all available data and a written summary of the discrepant points of view. The third clinician acts as a tie-breaker. Since there are at least 9 schizophrenia loci, it was expected that about 10% of these patients would be defective at SCZD7.

Method of Polymorphism ID.

Polymorphisms were initially detected by sequencing the relevant exon. Sequencing in the second direction was performed routinely for all exonic regions. In addition, the redundancy of the study, sequencing 48 schizophrenia and 48 Panic Disorder Syndrome samples, assures that the same polymorphism is detected in multiple samples. We did not systematically include control DNA, reasoning that most exons would be normal at most positions, and that a mutation at on position would be evident on comparison to the BION1 cDNA sequence.

Exons Scanned for Polymorphisms in Panic Disorder Syndrome Samples.

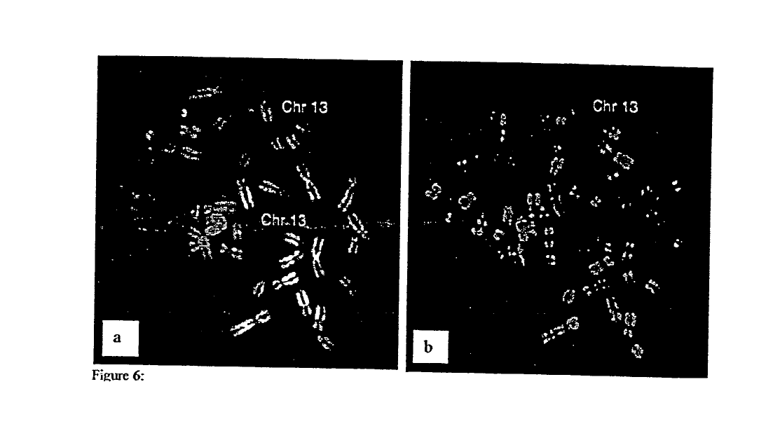

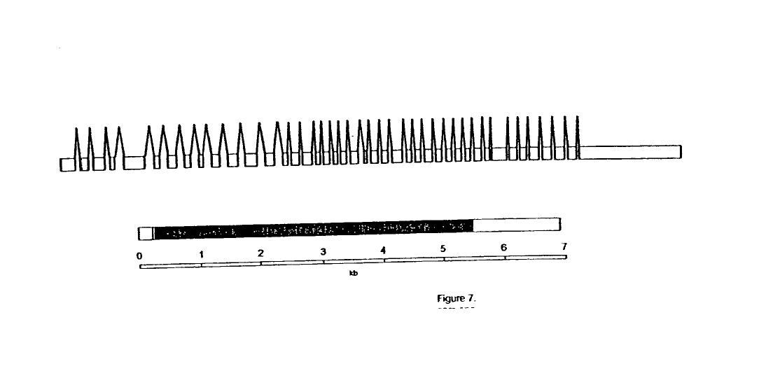

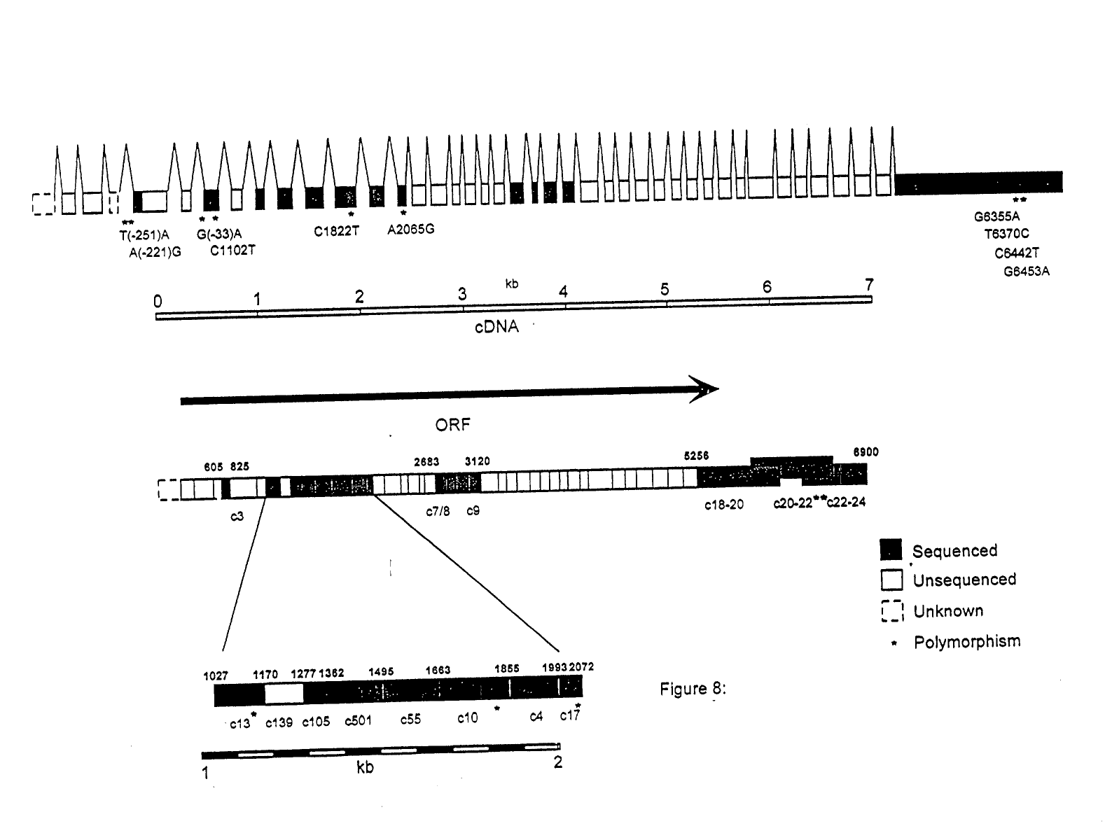

A total of 13 exons have been scanned, as well as the 3′ UTR, for 48 Panic Disorder Syndrome samples. This represents 1405 bp out of the 5231 bp coding sequence (27%) and 3050 bp out of the 6942 bp cDNA (44%). We found 2 exonic, 3 intronic, and 4 3′UTR polymorphisms (Table 2) (see FIG. 8).

TABLE 2
Polymorphisms found in Panic Disorder Syndrome gDNA samples

| | | Allele Frequency | | |
|---|---|---|---|---|
| Exon | Polymorphism ID | A/A* | A/B* | B/B* |
| C3 | T(−251)A† | 2 | 14 | 12 |
| | T(−221)C† | 12 | 14 | 2 |
| C13 | G(−33)A† | 10 | 21 | 9 |
| C10 | C1822T | 24 | 13 | 11 |
| C17 | A2065G | 39 | 4 | 0 |
| 3′UTR | G6355A | 3 | 4 | 4 |
| | T6370C | 11 | 11 | 21 |
| | C6442T | 24 | 10 | 10 |
| | G6453A | 43 | 0 | 1 |

*By convention, A is the nucleotide found in the BION1 cDNA, and it is given first in the polymorphism ID. B is the polymorphic nucleotide, and is given last. For intronic polymorphisms, the order is arbitrary. The number given in the polymorphism ID is the position on the cDNA of the polymorphic nucleotide.
†For intronic polymorphisms (indicated by the minus sign), the number refers to number of base-pairs from the nearest intron/exon boundary. For polymorphisms within the cDNA, nucleotide number of the BION1 cDNA is given.

Exons Scanned for Polymorphisms in Schizophrenia Samples.

The same 13 exons were scanned with the 48 Schizophrenia patient DNAs. This represents 1405 bp out of the 5231 bp coding sequence (27%). We found the same 2 exonic polymorphisms seen in the Panic Disorder samples, plus one novel polymorphism not seen in the previous scan (Table 3) (see FIG. 8).

TABLE 3
Polymorphisms found in NIMH Schizophrenic gDNA samples

| | | Allele Frequency | | |
|---|---|---|---|---|
| Exon | Polymorphism ID | A/A* | A/B* | B/B* |
| C13 | C1102T | 32 | 1 | 0 |
| C10 | C1822T | 28 | 18 | 1 |
| C17 | A2065G | 34 | 3 | 0 |

Polymorphisms Identified.

A2065G was found to be heterozygous in 3 schizophrenia samples (Repository IDs 90C00517, 90C03310, 90C03592) out of 37 (8%). This polymorphism was present in 4/43 (9.5%) of Panic Disorder Syndrome samples, so this polymorphism is consistently rare in both disorders. Interestingly, this consistency was not true when considering C1822T. In schizophrenia samples, this polymorphic nucleotide is T in only 1 sample (Repository ID 90C00518) out of 47 (2%). When the same polymorphism is examined in Panic Disorder Syndrome patients, 11/48 (23%) show a T at this position. The C1822T and A2065G polymorphisms were confirmed by sequencing the relevant exon in both directions. The intronic and 5′UTR polymorphisms were sequenced in Panic Disorder Syndrome samples, and not schizophrenia samples. C1102T was only seen in 1 patient with schizophrenia (Repository ID 90C02418), and no Panic Disorder Syndrome samples. Although this difference was seen in a rerun of the same sample, sequencing in the second direction was not done, so the identification of this polymorphism is tentative.

Confirmation of Polymorphisms.

In cases where the polymorphism was found to have generated or destroyed a restriction endonuclease cleavage site, then the existence of the polymorphism could be confirmed by performing a restriction digest on the relevant PCR product. This was the case for C1822T, where the polymorphic nucleotide led to the destruction of a TaqI site. TaqI digestion and agarose gel analysis confirmed the prediction that sequence analysis allowed us to make.

### REFERENCES

Andreasen, N. C. (1995) Symptoms, signs, and diagnosis of schizophrenia. Lancet 346:477-481.

Blouin, J-L et al. (1998) Schizophrenia susceptibility loci on chromosomes 13q32 and 8p21. Nature Genetics 20:70-73.

Brzustowicz, L. M. et al. (2000) Location of a Major Susceptibility Locus for Familial Schizophrenia on Chromosome 1q21-q22. Science 288:678-682

Knowles et al. (1998) Results of a genome-wide screen for panic disorder. Am J Med Genet (Neuropsychiatr Genet) 81:139-147.

Lee, J-H et al. (1999) Cloning of a novel four repeat protein related to voltage-gated sodium and calcium channels. FEBS Letters 445:231-236.

Millar, J. K. et al. (2000) Disruption of two novel genes by a translocation co-segregating with schizophrenia. Hum Mol Genet. 9:1415-1423.

Weissman, M. S et al. (2000) Potential Panic Disorder Syndrome: Clinical and Genetic Linkage Evidence. American Journal of Medical Genetics (Neuropsychiatric Genetics) 96:24-35

1. An isolated and purified polynueleotide which encodes human BION1 which has an amino acid sequence as shown in SEQ ID NO:1 or a polymorphic variant found in a schizophrenic, Bipolar Disorder, or Panic Disorder Syndrome patient.

2. The isolated and purified polynucleotide of claim 1 wherein the polynucleotide is a cDNA.

3. The isolated and purified polynucleotide of claim 1 wherein the polynucleotide is genomic DNA.

4. The isolated and purified polynucleotide of claim 1 wherein the polynucleotide is in a YAC clone.

5. The isolated and purified polynucleotide of claim 1 wherein the polynucleotide is in a BAC clone.

6. The isolated and purified polynucleotide of claim 4 wherein the YAC clone further comprises marker D135779.

7. The isolated and purified polynucleotide of claim 1 wherein the polynucleotide comprises the amino acid-coding sequence shown in SEQ ID NO:2.

8. The isolated and purified polynucleotide of claim 1 comprising the sequence shown in SEQ ID NO:2.

9. The isolated and purified polynucleotide of claim 1 wherein the polynucleotide is a 6.2 kb transcript as measured on Northern blots.

10. The isolated and purified polynucleotide of claim 1 which comprises a polymorphism shown in Table 2.

11. The isolated and purified polynucleotide of claim 1 which comprises a polymorphism shown in Table 3.

12. A method of determining susceptibility to Schizophrenia, Bipolar Disorder, or Panic Disorder Syndrome, comprising:
determining a nucleotide at a defined location in a BION1 gene of a human;
comparing the nucleotide at the defined location of the human to that of an affected family member having a polymorphism at the nucleotide;
identifying the human as susceptible to schizophrenia, bipolar disorder, or panic disorder if the determined nucleotide contains the polymorphism found in the affected family member.

13. The method of claim 12 wherein the family member is schizophrenic.

14. The method of claim 12 wherein the family member has Bipolar Disorder.

15. The method of claim 12 wherein the family member has Panic Disorder Syndrome.

16. The method of claim 12 wherein the polymorphism is identified in Table 2 or Table 3.

17. A method of determining susceptibility to Schizophrenia, Bipolar Disorder, or Panic Disorder Syndrome comprising:
determining a nucleotide at a defined location in a BION1 gene of a human;
if the determined nucleotide is a polymorphism previously identified as associated with Schizophrenia, Bipolar Disorder, or Panic Disorder Syndrome, identifying the human as susceptible to Panic Disorder Syndrome, Bipolar Disorder, or Schizophrenia.

18. An isolated and purified polynucleotide comprising at least 18 contiguous nucleotides of a human BION1 coding sequence, wherein said polynucleotide comprises at least one codon identified in Table 1, which codon is for an amino acid found in human BION1 proteins at the corresponding position.

19. An isolated and purified human BION1 protein comprising an amino acid sequence as shown in SEQ ID NO:1 or a polymorphic variant found in Schizophrenia, Bipolar Disorder, or Panic Disorder Syndrome patients.

20. An isolated and purified human BION1 polypeptide comprising at least 6 amino acids of a human BION1 protein, wherein said polypeptide comprises an amino acid sequence found in humans but not in rats as identified in Table 1.

21. An isolated and purified polynucleotide of a human BION1 gene, wherein said polynucleotide comprises a polymorphic nucleotide identified in Table 2 or Table 3.

22. A vector comprising the polynucleotide of claim 1.

23. A host cell comprising the vector of claim 22.

24. A method of producing human BION1 comprising:
culturing the host cell of claim 23 under conditions for expression of BION1 from the vector;
collecting BION1 protein from the cultured host cells or culture medium.

25. A method of screening test substances for candidates useful in treating Schizophrenia, Bipolar Disorder, or Panic Disorder Syndrome comprising:
contacting a human BION1 protein with a test substance;
determining which test substances bind to the human BION1 protein; wherein a test substance is identified as a candidate drug useful for treating Schizophrenia, Bipolar Disorder, or Panic Disorder Syndrome if it binds to human BION1.

26. The method of claim 25 wherein the human BION1 protein has a polymorphic amino acid identified in Table 2 or Table 3.
